(19) United States
(12) Patent Application Publication
(10) Pub. No.: US 2012/0029650 A1
(43) Pub. Date: Feb. 2, 2012

(54) MEDICAL DEVICE

(75) Inventor: Warwick J.M. Bruce, Lewisham (AU)

(73) Assignee: T.J. SMITH & NEPHEW LIMITED, Memphis, TN (US)

(21) Appl. No.: 13/127,400

(22) PCT Filed: Nov. 5, 2009
(86) PCT No.: PCT/GB09/02611

§ 371 Date: Oct. 6, 2011

(30) Foreign Application Priority Data
Nov. 5, 2008 (GB) 0820233.5

(51) Int. Cl. A61F 2/32

(52) U.S. Cl. 623/22.4

(57) ABSTRACT

An implant, comprising: a head having a central axis; and a stem having a stem axis, wherein the head central axis and the stem axis are not co-linear. A method for resurfacing a femoral head, comprising the steps of: providing an implant according to the present invention; and implanting the implant with reference to the centre of the femoral neck.

The present invention relates to medical devices, particularly hip implants.

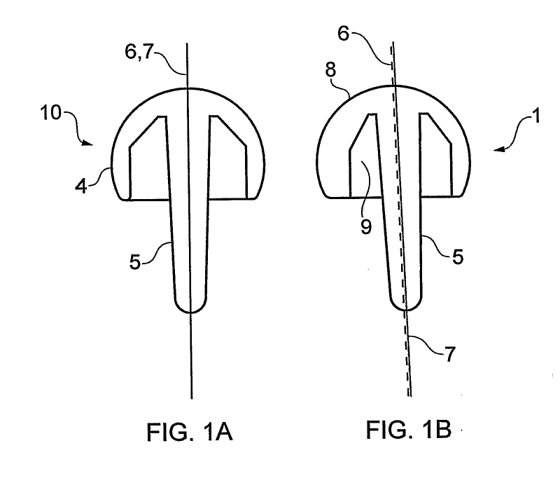

Known femoral head resurfacing designs have the stem axis and the femoral head axis concurrent. That is, the stem axis and the femoral head axis are co-linear (see FIG. 1A). Femoral anatomy varies significantly between patients. The centre of the femoral neck is often not the centre of the femoral head. Consequently, mismatches can occur when a femoral head is resurfaced with a conventional resurfacing design. Such mismatches mean that known resurfacing designs do not reproduce the natural biomechanics and stability of the joint. This can lead to many problems for the patient, ranging from discomfort to pain and ultimately to failure, necessitating revision surgery.

With known femoral head resurfacing designs there is also little intra-operative flexibility in positioning the centre of the femoral head once the stem has been prepared. Consequently, the mismatches cannot be rectified and the above problems are encountered.

According to an aspect of the present invention, there is provided an eccentric resurfacing head.

According to an aspect of the present invention, there is provided an implant, comprising: a head having a central axis; and a stem having a stem axis, wherein the head central axis and the stem axis are not co-linear.

According to an aspect of the present invention, there is provided an implant for resurfacing a femoral head, comprising: a head having a central axis; and a stem having a stem axis, wherein the head central axis and the stem axis are not co-linear.

According to an aspect of the present invention, there is provided an implant, comprising: a head having a bearing surface centre; and a stem having a stem axis, wherein the stem axis is offset compared to the head bearing surface centre.

According to an aspect of the present invention, there is provided an implant for resurfacing a femoral head, comprising: a head having a bearing surface centre; and a stem having a stem axis, wherein the stem axis is offset compared to the head bearing surface centre.

According to an aspect of the present invention, there is provided a resurfacing femoral head and stem, wherein the stem axis is offset compared to the bearing surface centre.

According to an aspect of the present invention, there is provided an implant comprising a head having a bearing surface and a recess inside the head with a stem, the stem and recess having an axis, wherein the stem/recess axis and the head axis are offset from each other.

According to an aspect of the present invention, there is provided an implant for resurfacing a femoral head, comprising a head having a bearing surface and a recess inside the head with a stem, the stem and recess having an axis, wherein the stem/recess axis and the head axis are offset from each other.

According to an aspect of the present invention, there is provided an implant, comprising: a head having an external surface defining a bearing surface, the bearing surface having a centre, and an internal surface defining a recess within the head; and a stem disposed on the internal surface of the head and having a stem axis, wherein the stem axis is offset compared to the head bearing surface centre.

According to an aspect of the present invention, there is provided an implant for resurfacing a femoral head, comprising: a head having an external surface defining a bearing surface, the bearing surface having a centre, and an internal surface defining a recess within the head; and a stem disposed on the internal surface of the head and having a stem axis, wherein the stem axis is offset compared to the head bearing surface centre.

According to an aspect of the present invention, there is provided an implant, comprising: a head having an external surface defining a bearing surface, the bearing surface having a centre, and an internal surface defining a recess within the head; and a stem disposed on the internal surface of the head and having a stem axis, wherein the stem axis is offset compared to the head bearing surface centre, and wherein the stem and the internal surface are concentric.

According to an aspect of the present invention, there is provided an implant for resurfacing a femoral head, comprising: a head having an external surface defining a bearing surface, the bearing surface having a centre, and an internal surface defining a recess within the head; and a stem disposed on the internal surface of the head and having a stem axis, wherein the stem axis is offset compared to the head bearing surface centre, and wherein the stem and the internal surface are concentric.

The offset between the stem axis and the head bearing surface centre may be any suitable amount. The offset may be an integer or non-integer amount. The offset may be in the range 1-10 mm. The offset may be in the range 1-5 mm. The offset may be in the range 1-3 mm. The offset may be around 1 mm. The offset may be around 2 mm. The offset may be around 3 mm.

Preferably, the internal geometry of the head remains symmetric. This may be achieved by thickening one wall of the head (see FIG. 1B). This allows the surgeon to prepare the bone and then decide where to position the centre of the bearing.

The stem axis may be parallel, or substantially parallel, to the head centre axis.

The stem may be angled. That is, the stem axis may be angled with respect to the head centre axis.

The implant may comprise a separable head and stem (i.e. two-part design). The stem may be assembled onto the head in use.

Preferably, the implant is a monobloc/single part comprising a head and a stem.

An advantage associated with monobloc embodiments of the present invention is that they are mechanically superior to two-component embodiments. For example, a two component device has fretting interfaces which can lead to wear. In contrast, a single component device does not have fretting interfaces and is therefore more resistant to wear.

Implants according to the present invention may comprise one or more bone in-growth surfaces.

Implants according to the present invention may be made of any suitable material. The implant may comprise metal. The implant may comprise metal alloy. The implant may comprise stainless steel. The implant may comprise titanium. The implant may comprise cobalt chrome.

According to the present invention, there is provided a method of implantation, comprising the steps of: providing an implant according to the present invention; and implanting the implant into a body.

According to the present invention, there is provided a method for resurfacing a femoral head, comprising the steps of: providing an implant according to the present invention; and implanting the implant with reference to the centre of the femoral neck.

An advantage associated with referencing the centre of the neck of the femur is that the risk of neck fracture is minimised or eliminated.

In use, the implant may be cemented.

In use, the implant may not be cemented.

Implants according to the present invention allow the surgeon final control on the bearing centre of the hip. This is important in restoring the natural biomechanics and stability of the joint.

Reference will now be made, by way of example, to the accompanying drawings, in which:

FIGS. 1A and 1B show cross-sections of a known femoral head resurfacing implant and a femoral head resurfacing implant according to an embodiment of the present invention, respectively;

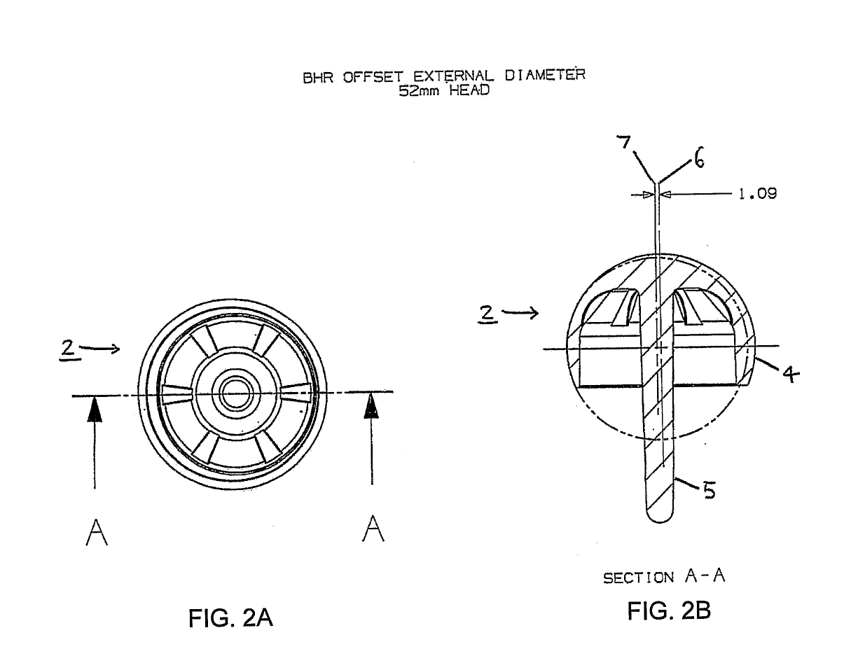

FIGS. 2A and 2B show a top plan view and a cross-section view of a femoral head resurfacing implant according to another embodiment of the present invention, respectively;

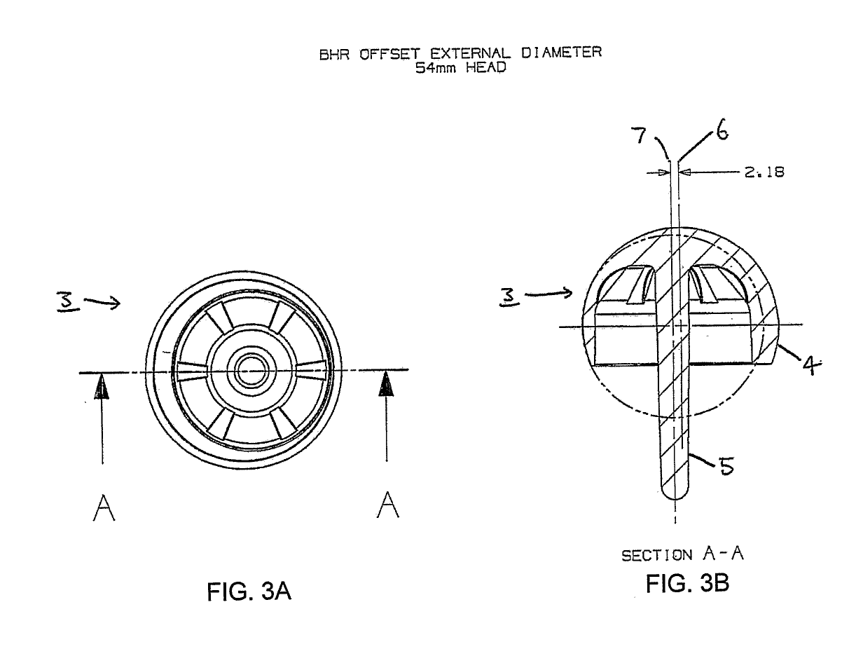

FIGS. 3A and 3B show a top plan view and a cross-section view of a femoral head resurfacing implant according to another embodiment of the present invention, respectively; and

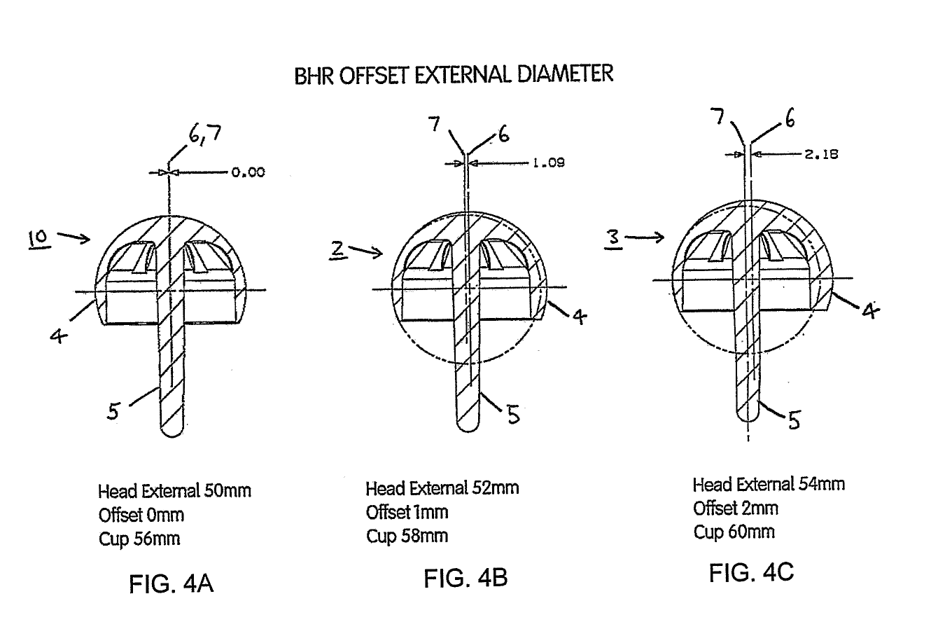

FIGS. 4A, 4B and 4C show cross-sections of a known femoral head resurfacing implant, the embodiment shown in FIG. 2B and the embodiment shown in FIG. 3B, respectively.

As shown in FIGS. 1A and 4A, for known femoral head resurfacing implants (10) the femoral head axis (6) and the stem axis (7) are co-linear. That is, the stem (5) is concentric to the head (4) centre. As shown in FIGS. 1B, 2A, 2B, 3A, 3B, 4B and 4C, for femoral head resurfacing implants (1-3) in accordance with the present invention, the femoral head axis (6) and the stem axis (7) are offset such that they are not co-linear. As shown in FIG. 1B, implants (1) according to an embodiment of the present invention comprise a stem (5) that is eccentric to the articulating surface (8) but concentric to the internal profile (9). FIGS. 2A, 2B and 4B show an implant (2) having an offset of approximately 1 mm. FIGS. 3A, 3B and 4C show an implant (3) having an offset of approximately 2 mm.

For example, the embodiment shown in FIG. 4A may have a head external diameter of around 50 mm, an offset of 0 mm, and be coupled with a cup having a diameter of around 56 mm. The embodiment shown in FIG. 4B may have a head external diameter of around 52 mm, an offset of around 1 mm, and be coupled with a cup having a diameter of around 58 mm. The embodiment shown in FIG. 4C may have a head external diameter of around 54 mm, an offset of around 2 mm, and be coupled with a cup having a diameter of around 60 mm.

1-11. (canceled)

12. A hip implant comprising:
a head having an external surface defining a bearing surface, the bearing surface having a center, and an internal surface defining a recess within the head; and
a stem disposed on the internal surface of the head and having a stem axis, wherein the stem axis is offset compared to the head bearing surface center.

13. A hip implant according to claim 12, wherein the stem and the internal surface are concentric.

14. A hip implant according to claim 13, wherein the offset is in the range 1-3 mm.

15. A hip implant according to claim 14, wherein the implant is for resurfacing a femoral head.

16. A hip implant according to claim 15, wherein the implant is a monobloc.

17. A hip implant comprising a head having a central axis and a stem having a stem axis, wherein the head central axis and the stem axis are not co-linear.

18. A hip implant according to claim 17, wherein the stem and the internal surface are concentric.

19. A hip implant according to claim 18, wherein the offset is in the range 1-3 mm.

20. A hip implant according to claim 19, wherein the implant is for resurfacing a femoral head.

21. A hip implant according to claim 20, wherein the implant is a monobloc.

22. A method for resurfacing a femoral head, comprising the steps of:
providing a hip implant with a head having an external surface defining a bearing surface, the bearing surface having a center, and an internal surface defining a recess within the head; and a stem disposed on the internal surface of the head and having a stem axis, wherein the stem axis is offset compared to the head bearing surface center; and
implanting the implant with reference to the center of the femoral neck.
